(19) United States
(12) Patent Application Publication
(10) Pub. No.: US 2026/0124082 A1
(43) Pub. Date: May 7, 2026

(54) ABSORBENT GARMENT

(71) Applicant: Shenzhen Lute Jiacheng Supply Chain Management Co., Ltd., Shenzhen (CN)

(72) Inventors: Lan Li, Shenzhen (CN); Qian Feng, Shenzhen (CN); Ping Wang, Shenzhen (CN)

(21) Appl. No.: 19/437,285

(22) Filed: Dec. 30, 2025

(30) Foreign Application Priority Data
Dec. 2, 2025 (CN) 202522580503.7

(51) Int. Cl. A61F 13/496; A41B 9/00; A41B 17/00; A61F 13/15; A61F 13/494; A61F 13/513

(52) U.S. Cl. CPC A61F 13/4963; A41B 9/004; A41B 17/00; A61F 13/49426; A61F 13/51305; A61F 2013/15463; A61F 2013/1591; A61F 2013/51355

(57) ABSTRACT

The present application discloses an absorbent garment comprising: a pants body, two leg openings, and a crotch region located between the two leg openings; a base layer combined with the pants body at least on both sides of the crotch region, wherein the base layer and the pants body enclose a receiving space, and the base layer is configured to face toward the wearer's crotch during wearing; the pants body has a first edge on one side of the crotch region, the base layer has a second edge corresponding to the first edge, and the first edge and the second edge extend into the receiving space and are sewn together by stitching within the receiving space; and an absorption layer located within the receiving space and combined with the base layer or the pants body.

### CROSS-REFERENCE TO RELATED APPLICATIONS

The present application claims priority to Chinese Patent Application No. 202522580503.7, filed on Dec. 2, 2025, the entire contents of which are incorporated herein by reference.

### TECHNICAL FIELD

The present application relates to the technical field of maternal and baby products, particularly to a absorbent garment.

### BACKGROUND

In recent years, absorbent garments have been increasingly accepted as an emerging product. Typically used by women during menstruation or by individuals with incontinence, absorbent garments can absorb bodily fluids and prevent leakage. Unlike disposable absorbent products such as sanitary napkins and diapers, absorbent garments are reusable and washable clothing.

An absorbent garment mainly consists of a pants body and an absorbent assembly. The method of securing and connecting these two components directly affects the user's wearing experience.

As disclosed in U.S. patent application Ser. No. 16/567,212, one existing method involves bonding the absorbent assembly to the pants body using adhesive. While this approach offers advantages in preventing leakage and side leakage for a certain period, as bodily fluids can be completely contained within the absorbent assembly, it performs poorly in terms of breathability. Additionally, the garment does not dry easily after washing. Moreover, the adhesive tends to degrade under repeated washing or conditions such as machine drying, resulting in a relatively short service life.

As disclosed in U.S. patent application Ser. No. 17/475,690, another existing method involves using a bound-edge stitching technique. Specifically, both sides of the crotch region of the pants body wrap around the absorbent assembly to form elastic bound edges, which are then stitched to the absorbent assembly. In this method, the stitching is directly exposed and faces the wearer's crotch. Furthermore, due to the elasticity of the bound edges, the pressure generated by the elasticity presses the stitching tightly against the skin of the wearer's crotch, leading to reduced wearing comfort.

Therefore, there is a need to provide an absorbent garment to address the above technical issues.

### SUMMARY

In view of the deficiencies in the prior art, the present application provides an absorbent garment, which can solve the problems of poor wearing comfort and breathability in existing absorbent garments.

In order to solve the above technical problem, one embodiment of this application proposes an absorbent garment comprises: a pants body having an upper edge for surrounding a wearer's waist during wearing, two leg openings configured to allow the wearer's legs to pass through, and a crotch region located between the two leg openings; a base layer combined with the pants body at least on both sides of the crotch region, wherein the base layer and the pants body enclose a receiving space, and the base layer is configured to face toward the wearer's crotch during wearing; the pants body has a first edge on one side of the crotch region, the base layer has a second edge corresponding to the first edge, and the first edge and the second edge extend into the receiving space and are sewn together by stitching within the receiving space; and an absorption layer located within the receiving space and combined with the base layer or the pants body.

In one embodiment, the first edge of the pants body is folded and extends into the receiving space.

In one embodiment, the stitching is concealed within the receiving space.

In one embodiment, the base layer comprises a first region and a second region, the first region is located at a middle position of the crotch region, the second region is located at side positions of the crotch region, and a liquid absorption rate per unit area of the first region is greater than that of the second region.

In one embodiment, the first region is configured to allow penetration therethrough and flow toward the absorption layer, and the second region at least partially uses a waterproof material and is configured to prevent lateral diffusion of liquid and prevent penetration of liquid to the absorption layer.

In one embodiment, the first region is a one-way moisture-conducting fabric configured to allow liquid to penetrate unidirectionally from the wearer's body to the absorption layer, and the second region comprises a hydrophobic base layer and a waterproof adhesive layer laminated, with the hydrophobic base layer being closer to the wearer's skin than the waterproof adhesive layer.

In one embodiment, the first region is a first portion of the base layer, the second region is a second portion of the base layer, the first portion has a third edge, the second portion has a fourth edge adjacent to the third edge, and the third edge and the fourth edge extend into the receiving space and are combined within the receiving space.

In one embodiment, the fourth edge of the second portion is folded and extends into the receiving space.

In one embodiment, the third edge and the fourth edge are sewn together by stitching or bonded by an adhesive.

In one embodiment, the absorbent garment further comprises a pants body waterproof layer disposed on at least a portion of a surface of the pants body facing the receiving space.

In one embodiment, a width of the absorption layer is smaller than a width of the crotch region, and edges of the absorption layer do not extend to edges of the crotch region.

In one embodiment, the absorption layer and the first portion are jointly combined with the second portion, the absorption layer has a fifth edge, and the third edge, the fourth edge, and the fifth edge extend into the receiving space and are combined within the receiving space.

In one embodiment, the third edge, the fourth edge, and the fifth edge are sewn together within the receiving space.

In one embodiment, the absorption layer is combined with an inner surface of the pants body, and a position where the absorption layer is combined with the pants body is located at a non-edge position of the crotch region.

In one embodiment, an extension range of the absorption layer does not exceed a coverage range of the first region.

In one embodiment, the base layer is combined with the pants body at least on left and right sides of the crotch region.

Another embodiment provided by the present application is: an absorbent garment comprises: a pants body having an upper edge for surrounding a wearer's waist during wearing, two leg openings configured to allow the wearer's legs to pass through, and a crotch region located between the two leg openings; a base layer combined with the pants body at least on both sides of the crotch region, wherein the base layer and the pants body enclose a receiving space, and the base layer is configured to face toward the wearer's crotch during wearing; an absorption layer located within the receiving space and combined with the base layer or the pants body; and a liquid barrier portion disposed on the base layer, wherein the liquid barrier portion is disposed at side positions of the crotch region near the leg openings and is configured to protrude toward the wearer's crotch.

In one embodiment, the liquid barrier portion is a liquid barrier cotton strip disposed on the base layer.

In one embodiment, the liquid barrier portion is bonded to the base layer by an adhesive.

In one embodiment, the liquid barrier portion is disposed on a surface of the base layer facing the receiving space.

In one embodiment, the liquid barrier portion includes a protruding portion and an extension portion. The protruding portion protrudes toward the wearer's crotch, and the extension portion is connected to the protruding portion and extends along a surface of the base layer. A thickness of the protruding portion is greater than a thickness of the extension portion.

In one embodiment, there are two extension portions, which extend respectively along two sides of the protruding portion.

In one embodiment, a width of the absorption layer is smaller than a width of the crotch region, and edges of the absorption layer do not extend to edges of the crotch region.

In one embodiment, an extension range of the absorption layer does not cover a location of the protruding portion.

In one embodiment, there are two liquid barrier portions, which are respectively disposed on the base layer at positions corresponding to two sides of the crotch region.

In one embodiment, the liquid barrier portion extends in a strip-like manner along the wearer's groin during wearing.

The absorbent garment according to an embodiment of the present application comprises: a pants body having an upper edge for surrounding a wearer's waist during wearing, two leg openings configured to allow the wearer's legs to pass through, and a crotch region located between the two leg openings; a base layer combined with the pants body at least on both sides of the crotch region, wherein the base layer and the pants body enclose a receiving space, and the base layer is configured to face toward the wearer's crotch during wearing; the pants body has a first edge on one side of the crotch region, the base layer has a second edge corresponding to the first edge, and the first edge and the second edge extend into the receiving space and are sewn together by stitching within the receiving space; and an absorption layer located within the receiving space and combined with the base layer or the pants body. In this manner, the first edge and the second edge extend into the receiving space and are sewn together by stitching within the receiving space. This stitching method provides better breathability, thereby avoiding the lack of breathability caused by adhesive bonding. It also prevents the issue of difficulty in drying after washing, as well as the problem of adhesive degradation due to washing or drying. Furthermore, since the first edge and the second edge extend into the receiving space and are sewn together within it, the stitching is not exposed and does not cause uncomfortable friction against the wearer's crotch. Consequently, wearing comfort is improved, drying after washing is facilitated, adhesive failure is avoided, and the service life of the garment is enhanced.

### BRIEF DESCRIPTION OF THE DRAWINGS

To further clarify the technical solutions in the embodiments of this application or the prior art, a brief introduction to the drawings that need to be used in the description of the embodiments or prior art will be provided below. It is obvious that the drawings described below are merely some embodiments of this application, and for those skilled in the art, additional drawings may be obtained based on the structures shown in these drawings without requiring inventive effort.

FIG. 1 is a schematic diagram of the front structure of an absorbent garment according to a first embodiment of the present application;

FIG. 2 is a schematic diagram of the inner structure of the crotch region of the absorbent garment according to the first embodiment of the present application;

FIG. 3 is a cross-sectional schematic diagram of the absorbent garment according to the first embodiment of the present application taken along the crotch region;

FIG. 4 is a schematic diagram of the layered structure of a second region of a base layer in the absorbent garment according to the first embodiment of the present application;

FIG. 5 is a schematic diagram of the front structure of an absorbent garment according to a second embodiment of the present application;

FIG. 6 is a schematic diagram of the inner structure of the crotch region of the absorbent garment according to the second embodiment of the present application;

FIG. 7 is a cross-sectional schematic diagram of the absorbent garment according to the second embodiment of the present application taken along the crotch region.

The realization, functional characteristics, and advantages of the objectives of this application will be further explained in combination with the embodiments, with reference to the drawings.

### DETAILED DESCRIPTION OF THE EMBODIMENTS

In this application, the terms “provided,” “disposed,” and “connected” should be broadly understood. For example, they can refer to fixed connections, detachable connections, or integrated structures. They may indicate mechanical connections or electrical connections, direct connections, or indirect connections through an intermediary, or they may refer to internal communication between two devices, components, or parts. A person skilled in the art can understand the specific meaning of these terms in this application according to the context.

The terms “center,” “longitudinal,” “transverse,” “length,” “width,” “thickness,” “top,” “bottom,” “front,” “back,” “left,” “right,” “vertical,” “horizontal,” “inner,” “outer,” “radial,” and “circumferential” indicate directional or positional relationships based on the orientation or positional relationships shown in the figures. These terms are used solely to simplify and clarify the description of the present application and should not be interpreted as limiting the invention to a specific orientation, construction, or operation.

Additionally, the terms “first” and “second” are used only for descriptive purposes and should not be understood as indicating relative importance or implying the number of features. Therefore, a “first” or “second” feature may explicitly or implicitly include one or more of that feature. In the description of this application, “multiple” means at least two, such as two or three, unless otherwise explicitly specified.

Moreover, the above terms, in addition to indicating directional or positional relationships, may also have other meanings. For example, the term “top” may also refer to attachment or connection relationships. A person skilled in the art can understand these terms according to the specific context.

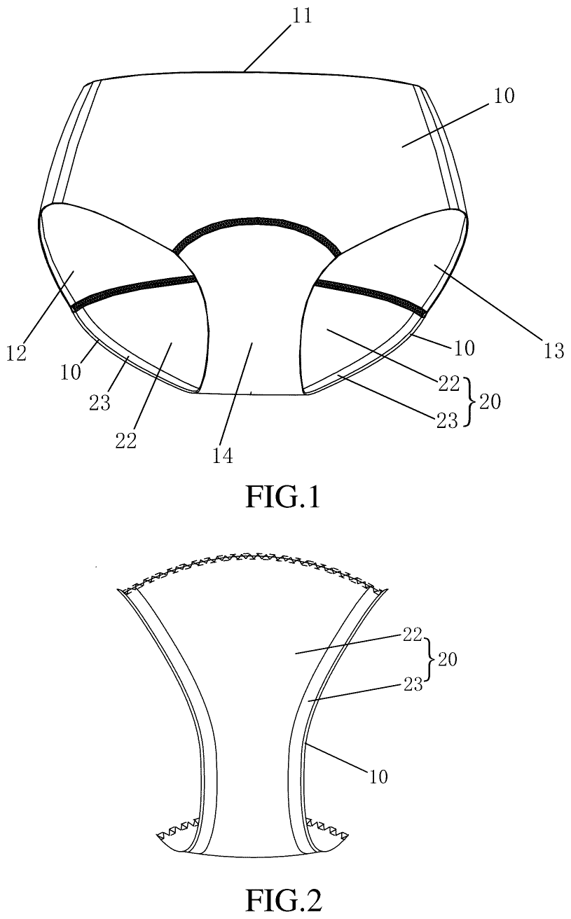

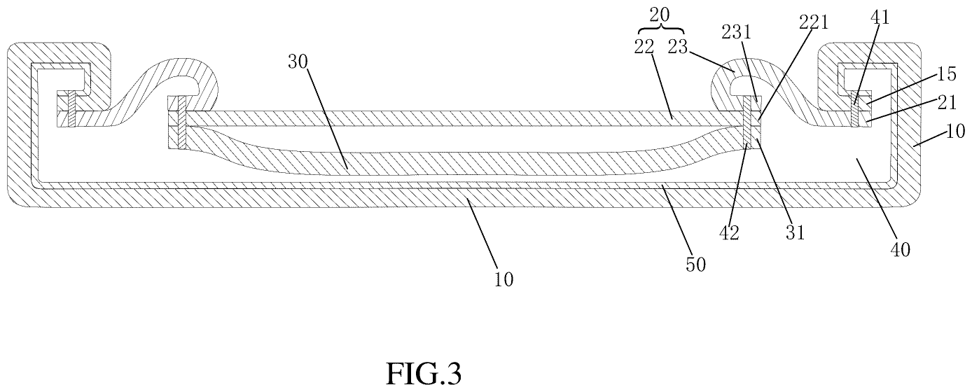

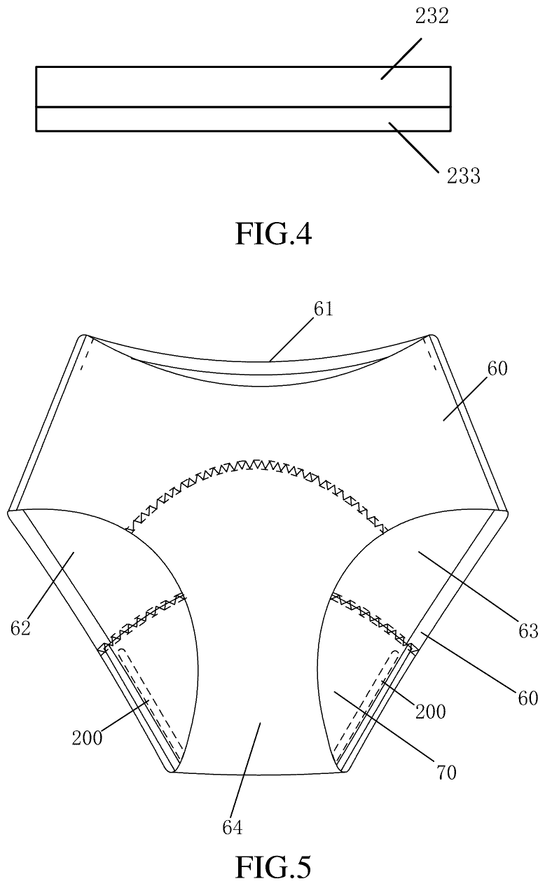

Please refer to FIGS. 1-4. FIG. 1 is a schematic diagram showing the front structure of an absorbent garment according to a first embodiment of the present application. FIG. 2 is a schematic diagram showing the inner structure of the crotch region of the absorbent garment according to the first embodiment. FIG. 3 is a cross-sectional schematic diagram of the absorbent garment according to the first embodiment taken along the crotch region. FIG. 4 is a schematic diagram showing the laminated structure of a second region of a base layer in the absorbent garment according to the first embodiment.

As shown in FIG. 2, in the first embodiment of the present application, the absorbent garment may include a pants body 10, a base layer 20, and an absorption layer 30.

As shown in FIG. 1, the pants body 10 has an upper edge 11 for surrounding a wearer's waist during wearing, two leg openings 12 and 13 configured to allow the wearer's legs to pass through, and a crotch region 14 located between the two leg openings 12 and 13.

The pants body 10 is made, at least in the crotch region 14, from a functional fabric that is breathable and moisture-wicking, such as Modal, Lycra, or cotton material with a honeycomb weave, to further enhance overall comfort.

As shown in FIGS. 1, 2, and 3, the base layer 20 is combined with the pants body 10 at least on both sides of the crotch region 14. The base layer 20 and the pants body 10 enclose a receiving space 40, and the base layer 20 is configured to face toward the wearer's crotch during wearing. In one embodiment, the base layer 20 is combined with the pants body 10 at least on the left and right sides of the crotch region 14. The “both sides of the crotch region 14” refer to the left and right sides respectively near the left and right leg openings 12 and 13.

As shown in FIG. 3, the pants body 10 has a first edge 15 on one side of the crotch region 14, and the base layer 20 has a second edge 21 corresponding to the first edge 15. The first edge 15 and the second edge 21 extend into the receiving space 40 and are sewn together by stitching 41 within the receiving space 40. The “one side edge of the crotch region 14” refers to either the left or right side. In this embodiment, both sides of the crotch region 14 are sewn using the same method described above. It should be understood that, in some embodiments, only one side may employ the aforementioned stitching method, while the other side may use a different stitching method or an adhesive bonding method, which is not limited in the embodiments of the present application.

As shown in FIG. 3, the absorption layer 30 is located within the receiving space 40 and combined with the base layer 20. The absorption layer 30 may be composed of one or more materials selected from terry cloth, dust-free paper, fluff pulp, superabsorbent polymer (SAP), and hydrophilic non-woven fabric. In some embodiments, the absorption layer 30 may also be combined with the pants body 10. For details, refer to the description of the embodiments below.

As shown in FIG. 3, in one embodiment, the first edge 15 of the pants body 10 is folded and extends into the receiving space 40. This ensures a smooth outer surface at the joint of the pants body, preventing discomfort in the crotch region.

As shown in FIG. 3, in one embodiment, the stitching 41 is concealed within the receiving space 40. By sewing the first edge 15 of the pants body 10 and the second edge 21 of the base layer 20 together within the receiving space 40, the stitching 41 is fully contained and hidden inside this space. This internal enclosed stitching structure provides multiple benefits: First, it ensures the stitching is completely isolated from the wearer's skin, fundamentally eliminating any potential friction from the stitches and thereby enhancing skin-contact comfort. Second, compared to large-area adhesive bonding, the stitching itself does not form a sealed barrier, preserving breathable pathways in this area. This allows for faster drying after washing and improves the feeling of dryness during wear. Finally, the connection method via stitching offers far superior mechanical strength and washing durability compared to adhesive bonding, effectively overcoming the issue of adhesive failure.

As shown in FIGS. 1, 2, and 3, in one embodiment, the base layer 20 includes a first region 22 and a second region 23. The first region 22 is located at a middle position of the crotch region 14, while the second region 23 is located at side positions of the crotch region 14. A liquid absorption rate per unit area of the first region 22 is greater than that of the second region 23. In this manner, due to the liquid wicking effect, liquid is concentrated in the first region 22 at the middle position of the crotch region 14, where it is absorbed and directed to the absorption layer 30, thereby reducing the risk of side leakage. In one embodiment, the coverage area of the first region 22 accounts for more than 90% of the total coverage area of the base layer 20, while the coverage area of the second region 23 accounts for less than 10% of the total coverage area of the base layer 20. There may be two second regions 23, which may be located on the left and right sides of the first region 22, respectively.

As shown in FIGS. 1, 2, and 3, in one embodiment, the first region 22 is configured to allow penetration therethrough and flow toward the absorption layer 30. The second region 23 at least partially uses a waterproof material and is configured to prevent lateral diffusion of liquid and prevent penetration of liquid to the absorption layer 30. The base layer 20 adopts a zoned design, with its core functions being directional liquid guidance and three-dimensional leakage prevention. The centrally located first region 22 serves as a guiding zone. It employs a unidirectional moisture-wicking fabric 22, which can quickly wick liquids such as menstrual fluid from the skin surface and direct them to the underlying absorption layer 30. Meanwhile, leveraging its unidirectional moisture-wicking property, it effectively reduces liquid backflow, maintaining relative dryness on the skin-contact surface.

As shown in FIGS. 1, 2, and 3, in one embodiment, the first region 22 is a unidirectional moisture-wicking fabric 22, configured to allow liquid to penetrate unidirectionally from the wearer's body to the absorption layer 30. In a specific example, the unidirectional moisture-wicking fabric 22 is a lightweight knitted fabric with a basis weight ranging from 95 g/m² to 110 g/m². Optionally, the unidirectional moisture-wicking fabric 22 has a rib-jacquard structure formed from hydrophobic polymer yarns (e.g., polyester), utilizing its specific knit construction to generate a capillary effect, thereby achieving the unidirectional moisture-wicking function. It should be understood that the unidirectional moisture-wicking fabric 22 is not limited to the aforementioned specific composition. It can be any functional fabric capable of achieving unidirectional liquid transport via capillary action, differential wettability, or a combination thereof. For example, the unidirectional moisture-wicking fabric 22 may be a double-layer composite structure, with its skin-facing layer comprising hydrophobic fibers such as fine-denier polyester or polypropylene, and its body-facing layer comprising hydrophilic fibers such as cotton or rayon. Alternatively, the unidirectional moisture-wicking fabric 22 may also be obtained by applying differential hydrophilic/hydrophobic finishing treatments to the inner and outer surfaces of a single-layer fabric.

As shown in FIG. 4, the second region 23 comprises a hydrophobic base layer 232 and a waterproof adhesive layer 233 arranged in a laminated configuration, with the hydrophobic base layer 232 being closer to the wearer's skin than the waterproof adhesive layer 233. The second regions 23 located on the sides constitute leakage prevention zones. Their laminated structure (the hydrophobic base layer 232 and the waterproof adhesive layer 233) works in synergy: the hydrophobic base layer 232, which is closer to the skin, effectively prevents liquid from spreading over its surface, forcing the liquid to concentrate toward the central guiding zone; while the waterproof adhesive layer 233 acts as a further barrier, preventing any potential penetrated liquid from wetting the pants body 10. This mechanism of zoned, coordinated operation significantly enhances the reliability of side leakage prevention while ensuring efficient absorption. The hydrophobic base layer 232 is a non-woven or woven fabric possessing hydrophobic properties. The waterproof adhesive layer 233 is a continuous waterproof barrier layer, its material selected from thermoplastic polyurethane (TPU) film, polyethylene (PE) film, or waterproof polyurethane (PU) coating. This layer is combined with the hydrophobic base layer 232 via a thermal lamination process, forming a barrier that, due to its dense and pore-free microstructure, completely blocks liquid penetration to the pants body 10.

As shown in FIGS. 1-3, in one embodiment, the first region 22 is a first portion 22 of the base layer 20, and the second region 23 is a second portion 23 of the base layer 20. The first portion 22 has a third edge 221, and the second portion 23 has a fourth edge 231 adjacent to the third edge 221. The third edge 221 and the fourth edge 231 extend into the receiving space 40 and are combined within the receiving space 40. In other embodiments, the base layer 20 may be a single, integral piece. For details, refer to the description of a second embodiment below.

As shown in FIG. 3, in one embodiment, the fourth edge 231 of the second portion 23 is folded and extends into the receiving space 40. This ensures a smooth outer surface at the joint of the pants body, preventing discomfort in the crotch region.

As shown in FIG. 3, in one embodiment, the third edge 221 and the fourth edge 231 are sewn together by stitching 42. In some other embodiments, the third edge 221 and the fourth edge 231 may also be bonded by an adhesive.

In one embodiment, the absorbent garment further comprises a pants body waterproof layer 50 disposed on at least a portion of a surface of the pants body 10 facing the receiving space 40. Optionally, the pants body waterproof layer 50 covers the entire inner surface of the crotch region 14.

In one embodiment, a width of the absorption layer 30 is smaller than a width of the crotch region 14, and edges of the absorption layer 30 do not extend to edges of the crotch region 14. In this manner, since the absorption layer 30 does not extend to the edges of the crotch region 14, the risk of side leakage can be reduced.

In one embodiment, the absorption layer 30 and the first portion 22 are jointly combined with the second portion 23. The absorption layer 30 has a fifth edge 31, and the third edge 221, the fourth edge 231, and the fifth edge 31 extend into the receiving space 40 and are combined within the receiving space 40.

In one embodiment, the third edge 221, the fourth edge 231, and the fifth edge 31 are sewn together by stitching 42 within the receiving space 40.

In one embodiment, an extension range of the absorption layer 30 does not exceed a coverage range of the first region 22.

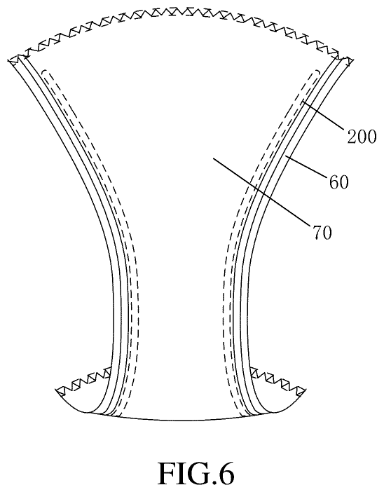

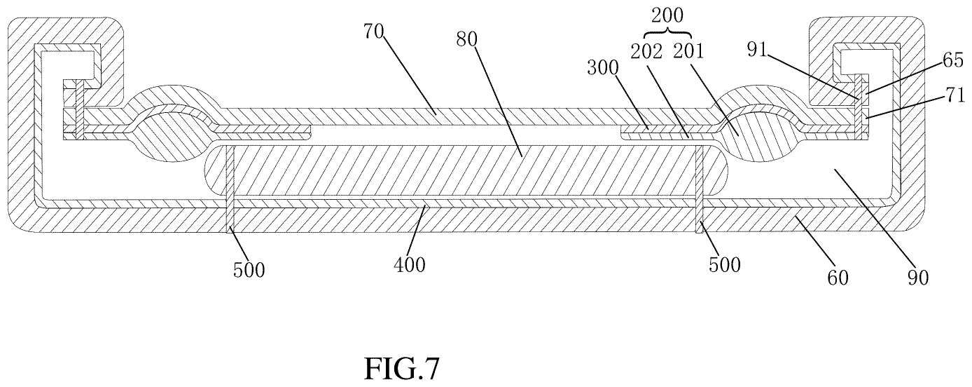

Please refer to FIGS. 5-7. FIG. 5 is a schematic diagram showing the front structure of an absorbent garment according to a second embodiment of the present application. FIG. 6 is a schematic diagram showing the inner structure of the crotch region of the absorbent garment according to the second embodiment. FIG. 7 is a cross-sectional schematic diagram of the absorbent garment according to the second embodiment taken along the crotch region.

As shown in FIGS. 5, 6, and 7, in the second embodiment of the present application, the absorbent garment may include a pants body 60, a base layer 70, and an absorption layer 80.

As shown in FIG. 5, the pants body 60 has an upper edge 61 for surrounding a wearer's waist during wearing, two leg openings 62 and 63 configured to allow the wearer's legs to pass through, and a crotch region 64 located between the two leg openings 62 and 63.

As shown in FIGS. 5, 6, and 7, the base layer 70 is combined with the pants body 60 at least on both sides of the crotch region 64. The base layer 70 and the pants body 60 enclose a receiving space 90, and the base layer 70 is configured to face toward the wearer's crotch during wearing. The pants body 60 has a first edge 65 on one side of the crotch region 64, and the base layer 70 has a second edge 71 corresponding to the first edge 65. The first edge 65 and the second edge 71 extend into the receiving space 90 and are sewn together by stitching 91 within the receiving space 90.

As shown in FIG. 7, the absorption layer 80 is located within the receiving space 90 and combined with the pants body 60.

As shown in FIG. 7, in one embodiment, the first edge 65 of the pants body 60 is folded and extends into the receiving space 90. In this manner, a smooth outer surface at the joint between the pants body and the base layer can be ensured, thereby improving wearing comfort.

As shown in FIG. 7, in one embodiment, the stitching 91 is concealed within the receiving space 90.

As shown in FIG. 7, in one embodiment, a width of the absorption layer 80 is smaller than a width of the crotch region 64, and edges of the absorption layer 80 do not extend to edges of the crotch region 64.

In one embodiment, the absorption layer 80 is combined with an inner surface of the pants body 60, and a position where the absorption layer 80 is combined with the pants body 60 is located at a non-edge position of the crotch region 64. The absorption layer 80 is sewn to the pants body 60 by stitching 500.

As shown in FIGS. 5, 6, and 7, in one embodiment, the absorbent garment further comprises a liquid barrier portion 200. The liquid barrier portion 200 is disposed on the base layer 70 and is located at side positions of the crotch region 64 near the leg openings 62, 63. The liquid barrier portion 200 is configured to protrude toward the wearer's crotch.

As shown in FIGS. 5, 6, and 7, in one embodiment, the liquid barrier portion 200 is a liquid barrier cotton strip 200 disposed on the base layer 70. Specifically, the liquid barrier portion 200 may be the liquid barrier cotton strip 200, whose operational mechanism is to form a three-dimensional embankment close to the body. When the wearer is in a supine or similar position, menstrual fluid may flow toward the thigh roots due to gravity. In such cases, the liquid barrier cotton strip 200 protruding from the surface of the base layer 70 can physically block the lateral spread of the liquid, confining it within the central absorption area enclosed by the liquid barrier portion 200 and the base layer 70, thereby significantly enhancing leakage protection during nighttime or while lying down. The liquid barrier cotton strip 200 is preferably made of a soft, highly elastic material, which provides effective blockage without creating hard pressure on the skin, ensuring wearing comfort.

As shown in FIGS. 5, 6, and 7, in one embodiment, the liquid barrier cotton strip 200 is bonded to the base layer 70 by an adhesive 300. For example, the liquid barrier portion 200 is bonded to the base layer 70 using hot-melt adhesive. It should be understood that, in other embodiments, the liquid barrier cotton strip may also be sewn onto the base layer 70 by stitching, which is not limited in the embodiments of the present application.

As shown in FIG. 7, in one embodiment, the liquid barrier cotton strip 200 is disposed on a surface of the base layer 70 facing the receiving space 90. In other embodiments, the liquid barrier portion may also be disposed on a side of the base layer 70 facing the wearer's crotch.

As shown in FIG. 7, in one embodiment, the absorbent garment further comprises a pants body waterproof layer 400 disposed on at least a portion of a surface of the pants body 60 facing the receiving space 90.

As shown in FIG. 7, in one embodiment, the liquid barrier portion 200 includes a convex portion 201 and an extension portion 202. The convex portion 201 protrudes toward the wearer's crotch, and the extension portion 202 is connected to the convex portion 201 and extends along a surface of the base layer 70. A thickness of the convex portion 201 is greater than a thickness of the extension portion 202. For example, the thickness of the convex portion 201 may be between 3 mm and 6 mm, and the thickness of the extension portion may be between 1 mm and 2 mm.

As shown in FIG. 7, in one embodiment, there are two extension portions 202, which extend along the two sides of the convex portion 201, respectively. The function of the extension portion(s) 202 is to increase the overall contact area between the liquid barrier portion 200 and the base layer 70, thereby making the bonding or sewing of the liquid barrier portion 200 to the base layer 70 more secure.

As shown in FIG. 7, in one embodiment, an extension range of the absorption layer 80 does not cover the location of the convex portion 201. In this manner, liquid from within the absorption layer 80 does not backflow to the convex portion 201, reducing the leakage prevention burden on the liquid barrier portion 200.

As shown in FIG. 7, in one embodiment, there are two liquid barrier portions 200, which are respectively disposed on the base layer 70 at positions corresponding to the two sides of the crotch region 64.

As shown in FIGS. 5, 6, and 7, in one embodiment, the liquid barrier portion 200 extends in a strip-like manner along the wearer's groin during wearing.

The absorbent garment according to any embodiment of the present application is configured to be reusable and washable multiple times.

The absorbent garment according to an embodiment of the present application comprises: a pants body having an upper edge for surrounding a wearer's waist during wearing, two leg openings configured to allow the wearer's legs to pass through, and a crotch region located between the two leg openings; a base layer combined with the pants body at least on both sides of the crotch region, wherein the base layer and the pants body enclose a receiving space, and the base layer is configured to face toward the wearer's crotch during wearing; the pants body has a first edge on one side of the crotch region, the base layer has a second edge corresponding to the first edge, and the first edge and the second edge extend into the receiving space and are sewn together by stitching within the receiving space; and an absorption layer located within the receiving space and combined with the base layer or the pants body. In this manner, the first edge and the second edge extend into the receiving space and are sewn together by stitching within the receiving space. This stitching method provides better breathability, thereby avoiding the lack of breathability caused by adhesive bonding. It also prevents the issue of difficulty in drying after washing, as well as the problem of adhesive degradation due to washing or drying. Furthermore, since the first edge and the second edge extend into the receiving space and are sewn together within it, the stitching is not exposed and does not cause uncomfortable friction against the wearer's crotch. Consequently, wearing comfort is improved, drying after washing is facilitated, adhesive failure is avoided, and the service life of the garment is enhanced.

While certain features of the embodiments of the subject matter claimed have been illustrated and described herein, many modifications, substitutions, changes, and equivalents will now occur to those skilled in the art. Moreover, although several functional modules and their relationships have been described in detail, it is believed by those skilled in the art that some operations can be performed without using other functional modules, or other functions or relationships between functions can be established while still conforming to the subject matter claimed. Therefore, it is intended that the appended claims encompass all such modifications and variations that fall within the true spirit and scope of the embodiments of the claimed subject matter.

The above description only provides specific embodiments of the present application. It should be noted that for ordinary technicians in the technical field, without departing from the principles of the present application, several improvements and embellishments can also be made, which should also be regarded as falling within the protection scope of the present application.

What is claimed is:

1. An absorbent garment, wherein the absorbent garment comprises:
a pants body having an upper edge for surrounding a wearer's waist during wearing, two leg openings configured to allow the wearer's legs to pass through, and a crotch region located between the two leg openings;
a base layer combined with the pants body at least on both sides of the crotch region, wherein the base layer and the pants body enclose a receiving space, and the base layer is configured to face toward the wearer's crotch during wearing; the pants body has a first edge on one side of the crotch region, the base layer has a second edge corresponding to the first edge, and the first edge and the second edge extend into the receiving space and are sewn together by stitching within the receiving space; and
an absorption layer located within the receiving space and combined with the base layer or the pants body.

2. The absorbent garment of claim 1, wherein the first edge of the pants body is folded and extends into the receiving space.

3. The absorbent garment of claim 1, wherein the stitching is concealed within the receiving space.

4. The absorbent garment of claim 1, wherein the base layer comprises a first region and a second region, the first region is located at a middle position of the crotch region, the second region is located at side positions of the crotch region, and a liquid absorption rate per unit area of the first region is greater than that of the second region.

5. The absorbent garment of claim 4, wherein the first region is configured to allow penetration therethrough and flow toward the absorption layer, and the second region at least partially uses a waterproof material and is configured to prevent lateral diffusion of liquid and prevent penetration of liquid to the absorption layer.

6. The absorbent garment of claim 5, wherein the first region is a one-way moisture-conducting fabric configured to allow liquid to penetrate unidirectionally from the wearer's body to the absorption layer, and the second region comprises a hydrophobic base layer and a waterproof adhesive layer laminated, with the hydrophobic base layer being closer to the wearer's skin than the waterproof adhesive layer.

7. The absorbent garment of claim 4, wherein the first region is a first portion of the base layer, the second region is a second portion of the base layer, the first portion has a third edge, the second portion has a fourth edge adjacent to the third edge, and the third edge and the fourth edge extend into the receiving space and are combined within the receiving space.

8. The absorbent garment of claim 7, wherein the fourth edge of the second portion is folded and extends into the receiving space.

9. The absorbent garment of claim 7, wherein the third edge and the fourth edge are sewn together by stitching or bonded by an adhesive.

10. The absorbent garment of claim 1, wherein the absorbent garment further comprises a pants body waterproof layer disposed on at least a portion of a surface of the pants body facing the receiving space.

11. The absorbent garment of claim 1, wherein a width of the absorption layer is smaller than a width of the crotch region, and edges of the absorption layer do not extend to edges of the crotch region.

12. The absorbent garment of claim 7, wherein the absorption layer and the first portion are jointly combined with the second portion, the absorption layer has a fifth edge, and the third edge, the fourth edge, and the fifth edge extend into the receiving space and are combined within the receiving space.

13. The absorbent garment of claim 12, wherein the third edge, the fourth edge, and the fifth edge are sewn together within the receiving space.

14. The absorbent garment of claim 1, wherein the absorption layer is combined with an inner surface of the pants body, and a position where the absorption layer is combined with the pants body is located at a non-edge position of the crotch region.

15. The absorbent garment of claim 4, wherein an extension range of the absorption layer does not exceed a coverage range of the first region.

16. The absorbent garment of claim 1, wherein the base layer is combined with the pants body at least on left and right sides of the crotch region.

17. An absorbent garment, wherein the absorbent garment comprises:
a pants body having an upper edge for surrounding a wearer's waist during wearing, two leg openings configured to allow the wearer's legs to pass through, and a crotch region located between the two leg openings;
a base layer combined with the pants body at least on both sides of the crotch region, wherein the base layer and the pants body enclose a receiving space, and the base layer is configured to face toward the wearer's crotch during wearing;
an absorption layer located within the receiving space and combined with the base layer or the pants body; and
a liquid barrier portion disposed on the base layer, wherein the liquid barrier portion is disposed at side positions of the crotch region near the leg openings and is configured to protrude toward the wearer's crotch.

18. The absorbent garment of claim 17, wherein the liquid barrier portion is a liquid barrier cotton strip disposed on the base layer.

19. The absorbent garment of claim 17, wherein the liquid barrier portion is bonded to the base layer by an adhesive.

20. The absorbent garment of claim 17, wherein the liquid barrier portion is disposed on a surface of the base layer facing the receiving space.
